(19) United States
(12) Plant Patent Application Publication
(10) Pub. No.: US 2012/0246776 P1
(43) Pub. Date: Sep. 27, 2012

(54) Chrysanthemum named 'Parlez Vous Pink'

(50) ;

(75) Inventor: Celine Emmanuelle Veronique Pesteil, Chichester (GB)

(73) Assignee: Chrysanthemum Breeders Association Research B.V., ZG Valkenburg Z-H (NL)

(21) Appl. No.: 13/064,407

(22) Filed: Mar. 23, 2011

(51) Int. Cl. A01H 5/00

(52) U.S. Cl. PLT/291

(57) ABSTRACT

A new variety of Chrysanthemum plant named ‘Parlez Vous Pink’, characterized by its good uniform canopy of pink flowers. The new variety has a medium vigor, a free branching and uniform spreading habit, a good foliage presentation and flowers week 39 in natural season.

### LATIN NAME OF THE GENUS AND SPECIES

Botanical classification: Chrysanthemum×morifolium Ramat.

### VARIETY DENOMINATION

The new Chrysanthemum variety denomination is ‘Parlez Vous Pink’.

### BACKGROUND OF THE INVENTION

The present invention comprises a new and distinct cultivar of Chrysanthemum botanically known as Chrysanthemum×morifolium Ramat., and referred to by the cultivar name ‘Parlez Vous Pink’, identified as 32014-0 originated from a cross made in a controlled breeding program in Chichester, West Sussex, United Kingdom. The new cultivar was discovered and selected as a flowering plant in September 2006.

The female parent of the new variety is an unpatented variety called ‘Yutaka Red’ and the male parent is an undistributed, unpatented seedling identified as ‘31644-0’.

The new variety ‘Parlez Vous Pink’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Parlez Vous Pink’ is a garden type of Chrysanthemum having a uniformed domed capitulum of pink decorative flowers.

### Comparison with Female Parent

Plants of the new Chrysanthemum variety ‘Parlez Vous Pink’ are dissimilar to the female parent ‘Yutaka Red’ in plant habit and growth rate. In side-by-side comparisons in Chichester, West Sussex, United Kingdom, under commercial practice, plants of the new Chrysanthemum variety ‘Parlez Vous Pink’ differed from plants of the female parent ‘Yutaka Red’ in the following characteristics:

1. The new variety ‘Parlez Vous Pink’ produces pink decorative flowers whereas the female parent ‘Yutaka Red’ produces red decorative flowers.

2. Plants of the new variety ‘Parlez Vous Pink’ have larger and taller inflorescence than plants of the female parent ‘Yutaka Red’.

3. Plants of the new variety ‘Parlez Vous Pink’ have a more vigorous habit than plants of the female parent ‘Yutaka Red’.

### Comparison with Male Parent

Plants of the new Chrysanthemum variety ‘Parlez Vous Pink’ are similar to the male parent ‘31644-0’ in plant habit and growth rate. However, in side-by-side comparisons in Chichester, West Sussex, United Kingdom, under commercial practice, plants of the new Chrysanthemum variety ‘Parlez Vous Pink’ differed from plants of the male parent ‘31644-0’ in the following characteristics:

1. The new variety ‘Parlez Vous Pink’ produces pink decorative flowers whereas the male parent ‘31644-0’ produces white-purple single flowers.

2. Plants of the new variety ‘Parlez Vous Pink’ have smaller and shorter inflorescence than plants of the male parent ‘31644-0’.

### Comparison with Most Similar Variety

Plants of the new Chrysanthemum variety ‘Parlez Vous Pink’ are similar to plants of the variety ‘Zanmuaval’ (U.S. patent application Ser. No. 12/805,257) in plant habit and growth rate. However, in side-by-side comparisons in Chichester, West Sussex, United Kingdom, under commercial practice, plants of the new Chrysanthemum variety ‘Parlez Vouz Pink’ differed from plants of ‘Zanmuaval’ in the following characteristics:

1. The new variety ‘Parlez Vous Pink’ produces smaller decorative blooms than plants of ‘Zanmuaval’.

2. Ray-florets of the new variety ‘Parlez Vous Pink’ are becoming light pink as they mature to make a two tones flower, whereas ray-florets ‘Zanmuaval’ are evenly pink in all stages of blooming.

3. Plants of the new variety ‘Parlez Vous Pink’ have a rounder shape than the plants of ‘Zanmuaval’.

### BRIEF DESCRIPTION OF ILLUSTRATIONS

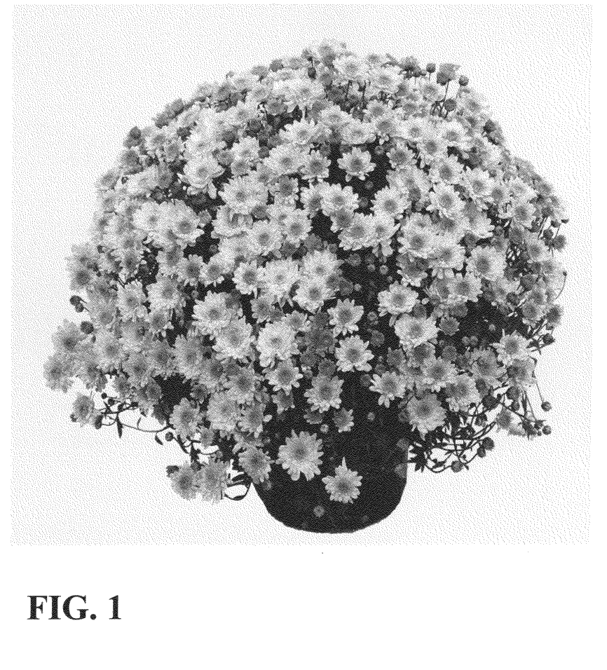

Typical specimens of the plant and flowers for the new Chrysanthemum variety ‘Parlez Vous Pink’ are shown in the accompanying photograph. The colors shown are as true as possible within the usual limits of this kind of illustration. FIG. 1 is a whole plant view of the new variety ‘Parlez Vous Pink’ grown in a pot. The plant shown in the illustration is around 120 days from the commencement of planting outdoors in natural season conditions.

### DETAILED BOTANICAL DESCRIPTION

The following description of the new Chrysanthemum variety ‘Parlez Vous Pink’ is of plants grown outside, natural season, in Chichester, West Sussex, United Kingdom in September 2010. The cultivar has not been observed under all possible environmental conditions. The phenotype may vary significantly with variations in the environment such as temperature, length of day and light intensity, without any variance in genotype. The commercial classification of the new variety is a garden Chrysanthemum.

Plants of the new variety have been grown successfully under temperature conditions averaging about 10° C. at night and about 10° C. to 25° C. during the day. The plants were grown outdoors in 4 liter pots using a loamless compost. No growth retardants were used.

To produce a commercial product, the plants don't need to be pinched. The typical container size for commercial growth is 4 liters. It has been observed that the shelf life of the new variety is about 28 days with a response time of about 10 weeks natural season. The new variety is suitable for a temperature range of 10° C. to 25° C.

The new variety may be produced as a garden pot plant. The following description is with respect to a plant produced as a garden pot plant. In the description of this new Chrysanthemum variety, color values have been taken from The Royal Horticultural Society Colour Chart, edition 2001.
Plant:
   Plant type.—Pot.
   Habit.—Domed, free branching and spreading.
   Height (cm).—35.
   Width (cm).—50.
   Stem.—Length (cm): 4 Diameter (cm): 0.5 -0.8 Texture: Rough Color: near Grey-Brown 199B.
   Branching characteristics.—Free branching and spreading.
   Lateral branches.—Length (cm): 15-20 Diameter (cm): 0.5-0.7 Texture: Slightly rough.
   Number of breaks from natural pinch.—9.
   Natural season flowering date.—week 39.
   Vigor.—Medium.
   Shelf life.—28 days.
   Life of blooms.—28 days.
   Disease (susceptibility/resistance observed).—None observed to date.
   Pest (susceptibility/resistance observed).—None observed to date.
   Drought and temperature (susceptibility/resistance observed).—None observed to date.
   Growth retardant type and treatment.—No treatment necessary. The plants were grown for 17 weeks in natural season conditions.
   Propagation.—Type: Vegetative propagation via stem cuttings Time to rooting: 12 days with soil temperatures of 21° C. Rooting habit: In 12 Long Days, a complete root system is developed.
Foliage:
   Number of leaves per lateral branch.—6-9.
   Compound or single.—Single.
   Arrangement of leaves.—Alternate. Shape of Leaf: Typically 5 lobed ovate. Size of Leaf: Width (cm): 2.5; Length (cm): 4 Leaf Apex: Acute Base: Corbate Attachment: Petioled Aspect: Slightly undulating Margin: Palmately lobed Surface characteristics: Top: Slightly pubescent Bottom: Slightly pubescent.
   Petiole.—Color: near Yellow-green 147A Length (cm): 1.1-1.3 Diameter (cm): 0.1-02 Surface: Slightly pubescent.
   Venation.—Net, prominent mid-veins at underside Color: upper side: near Green 137B. under side: near Green 137C.
   Color.—Mature Leaf, upper side: near Green137A; under side: near Yellow-green 147B Young Leaf, upper side: near Green 139A; under side: near Green 137B.
Inflorescence:
   Inflorescence appearance.—Matte.
   Inflorescence type.—Single.
   Inflorescence form.—Slightly cupped.
   Inflorescence shape.—Round.
   Flowering habit.—Cyme.
   Number of inflorescences per branch.—5-7.
   Depth of fully expanded blossoms (cm).—1.2.
   Diameter of fully expanded blossoms (cm).—4.
   Phyllaries.—Number: 20-24. Color: near Green143A Length (cm): 0.5-0.7 Width (cm): 0.1-0.3 Texture/Appearance: Shiny/slightly pubescent.
   Pedicel.—Length (cm): 2-4.5 Diameter (cm): 0.1 Angle from stem (degrees): 40 Color: near Yellow-green 147 B Surface: Slightly pubescent Habit: Slightly curving Strength: Medium/strong.
   Ray florets.—Form/Shape: Straight. Texture/Appearance: Matte. Number per flower: About 180 Length (cm): 0.5-1.3; Width (cm): 0.1-0.4 Apex: Rounded Base: Tubular Margin: Entire.
   Disc florets.—Form/Shape: Cylindrical Texture/Appearance: Matte Number per flower: 3-5 Length (cm): 0.3; Width (cm): 0.1 Diameter of disc (cm): 0.15.
   Fragrance.—None.
   Flower bud (at onset of color).—Length (cm): 0.4. Diameter (cm): 0.7 Form/Shape: Domed flat.
   General flower color.—1. Ray florets, upper side: Immature: Red-purple 65B Mature: Red-Purple 65D Older/Fading: Red 56C 2. Ray florets, under side: Immature: Red-purple 70A Mature: Red-purple 65C Older/Fading: Red 56C 3. Disc florets: Immature: Yellow-orange 14A Mature: Yellow-orange 14A Older/Fading: Yellow-orange 14A 6. Bud: near Red-purple 70B.
   Flower progression with age.—The flowers are becoming lighter with age.
Reproductive organs: Gynoecium present on Ray and Disc florets
   Ray florets per individual flower.—Pistillate. Pistil number: about 180 Stigma color: near Yellow-orange 14A Stigma shape: Forked Style color: near Yellow-green 154B Style length (cm): 0.5
   Disc florets per individual flower.—Pistillate Pistil number: 3-5 Stigma color: near Yellow-orange 14A Stigma shape: Forked Style color: near Yellow-green 154B Style length (cm): 0.5 .
   Androecium present on disc florets only.—Staminate. Stamen number: 3-5 Anther shape: Clubbed Anther color: near Yellow-orange 17B Pollen quantity: none.
   Seeds.—None observed.

What is claimed is:

1. A new and distinct variety of Chrysanthemum plant, substantially as described and illustrated herein.
